SEQUENCE LISTING (1 sequence)

SEQ ID NO: 1 - DNA, 1456 bp, Bovine
atgtcactacagatggtaacagtcggaaatagcatagccttaattcaaccaggcttctcgttaatgaattttgatgggcaagttttcttctttggccaaaaaggctggcccaagaggtcttgccccactggagttttccattttgaggtaaagcataatcatcttaaactgaagcctgcagttttctctaaggattcctgctaccttcctcctcttcgataccgggccacttgcacattcagcggccaacttggagtctgaaaagcatcagtacatcatccatggaggaaaaacaccaaacaatgagctttcagataagatttatgtgatgtctgttgtttccaagaacaacaaaaaagttacctttcgctgcacagagaaggacttggtaggagacattcctgaaggcagatatggtcattccattgatgtggtgtatagtcgggggaaaagtatgggtgttctctttggaggacggtcatacataccttctgcccaaagaaccacagagaaatggaacagtgtagctgactgcctgccccatgtcttcttggtggattttgaatttgggtgctctacgtcatacattcttccagaacttcaagatggactatcttttcatgtctccattgccagaaatgataccgtttatattttaggaggccattcacttgccaataacatccgccctgccaatctgtacagaataagggttgatctccccctgggtagcccagctgtggagtgcacagtcttgccaggaggaatctctgtctccagtgcaatcctgactcaaataagcaatgatgaatttgttattgttggtggctatcagcttgaaaatcaaaaaagaatggtctgtaacatcatctctttcaagtataacaagatagacattcttgagatggaaaccccagattggaccccagatattaagcacagcaagatatggtttggaagcaacatgggaaatggaactgttttcctcggcataccaggagacaataaacaggctgtttcagaagcattttacttctatacattgaaatgtgctgaagacgatgtgaacgaagatcagataactttgacaagtagtcagacatcaacagaagacccaggggactccactccctttgaagactcagaagaattttgcttcagcgcagaagcaaacagtttcgatggtgatgatgaatttgacacctacaatgaagatgatgaggaagatgagtctgagacaggctattggattacatgctgccctacttgtgatgtggatatcaatacgtgggtaccattttattcaactgagctcaacaagcctgccatgatctattgctctcatggagatggacattgggtccatgcccagtgtatggatctggcagaacgcaccacctcatccatctatcagaaggaagcaataaatattaytgtaacgagcatgtggagatag

(19) United States
(12) Patent Application Publication
(10) Pub. No.: US 2005/0183145 A1
(43) Pub. Date: Aug. 18, 2005

(54) Production of ungulates, preferably bovines that produce human immunoglobulins

(76) Inventors: Richard A. Goldsby, Leverett, MA (US); James M. Robl, Brandon, SD (US); Barbara A. Osborne, Leverett, MA (US)

Correspondence Address: CLARK & ELBING LLP, 101 FEDERAL STREET, BOSTON, MA, 02110, US

(21) Appl. No.: 11/011,711

(22) Filed: Dec. 14, 2004

Related U.S. Application Data
(63) Continuation of application No. 09/714,185, filed on Nov. 17, 2000, now abandoned.
(60) Provisional application No. 60/166,410, filed on Nov. 19, 1999.

(51) Int. Cl. A01K 67/027

(52) U.S. Cl. 800/15; 800/21

(57) ABSTRACT

The present invention relates to a method of producing an ungulate having both copies of the IgM heavy chain (mu) rag-1 and/or rag-2 gene eliminated from its genome. Animals which have IgM, rag-1 and/or rag-2 eliminated from their genome are unable to conduct the gene rearrangements that are necessary to generate the antigen receptors of B or T lymphocytes, and therefore will not develop native B or T cells. Because they are unable to produce B and T lymphocytes, these IgM, rag-1 or rag-2 ungulates cannot reject human hematopoietic stem cell preparations, and B and T lymphocytes which develop therefrom. Therefore, the present invention also involves injecting into IgM, rag-1 and/or rag-2 deficient ungulates, in utero or shortly after birth, human B and T lymphocytes whose immune systems produce human immunoglobulin that can be processed for therapeutic uses in humans.

### BACKGROUND OF THE INVENTION

I. Field of the Invention

This invention relates to a method for stably engrafted non-bovine (xenogeneic), preferably human B and T cells in ungulates, and other hoofed animals such as bovines, pigs, horses, sheep, buffalo and goats. The method of the present invention is particularly advantageous because it should result in cloned ungulates and other hoofed animals, e.g., bovines, that produce non-bovine, preferably human in lieu of endogenous antibodies. The invention more specifically relates to a method for producing IgM, Igα, E2A, EBF, BSAP, rag-1 or rag-2 knockout ungulates, that do not express endogenous immunoglobulins, which are engrafted with heterologous hematopoietic stem cells.

II. Description of the Related Art

One of the major impediments facing the development of in vivo therapeutic and diagnostic applications for antibodies in humans is the intrinsic immunogenicity of non-human immunoglobulins. For example, when immunocompetent human patients are administered therapeutic doses of rodent antibodies, the patients produce antibodies against the rodent immunoglobulin sequences; these human anti-mouse antibodies (HAMA) neutralize the therapeutic antibodies and can cause acute toxicity. Hence, it is desirable to produce human immunoglobulins that are reactive with specific antigens that are: pathogenic or contribute to pathogenic conditions, or are otherwise promising therapeutic and/or diagnostic targets.

Present technology for obtaining polyclonal human antibody for use in passive immunotherapy or prophylaxis involves collection of blood from thousands of human donors, pooling it and extracting human immunoglobulin. This technology producing human antibody or use in therapy has two major drawbacks. First, the supplies of human blood are too small to meet the demand for human immunoglobulin. Second, medical and ethical considerations preclude the deliberate immunization of human donors with a broad panel of microbes and other agents, many of which are potentially pathogenic, to assure that antibodies to these agents are present and of the highest practicable titer. There are no improvements to this current technology for obtaining polyclonal human antibody for passive immunotherapy that are likely to solve these important quantitative and qualitative problems.

Previous technology for generating monoclonal antibodies involved pre-exposing, or priming, an animal (usually a rat or mouse) with antigen, harvesting B-cells form that animal, and generating a library of hybridoma clones. By screening a hybridoma population for antigen binding specificity (idiotype) and also screening for immunoglobulin class (isotype), it is possible to select hybridoma clones that secrete the desired antibody. However, when these methods are applied for the purpose of generating human monoclonal antibodies, obtaining hybridum that produce human antibodies of predefined specificity is a serio8us technological obstacle.

The construction of animals that are transgenic for various forms, rearranged and unrearranged, of human immunoglobulin genes has been used to produce human antibodies in nonhuman species.

Transgenic animals which produce foreign immunoglobulin are well known in the art. For example, Lonberg et al. (U.S. Pat. Nos. 5,814,318; 5,877,397; 5,874,299; 5,789,650; 5,770,429; 5,661,016; 5,625,126; 5,545,806) disclose a method of producing transgenic non-human animals which produce human antibodies. The methods of Lonberg et al. involved either suppressing the endogenous immunoglobulin genes by using antisense polynucleotides and/or antiserum directed against endogenous immunoglobulins or inactivating both the endogenous light and heavy chain genes by homologous recombination. They next introduced sequences encoding the foreign immunoglobulin genes thereby producing a transgenic animal. The method of Lonberg et al. produces a variety of antibodies having various isotypes specific for a specific antigen.

Surani et al. (U.S. Pat. No. 5,545,807) also discloses a method for producing antibodies from transgenic animals. The method of Surani et al. involves using a host animal which lacks the genetic material relevant for encoding immunoglobulins. To this animal host, genetic material is added that encodes for heterologous unrearranged and rearranged immunoglobulin heavy and light chain of foreign origin capable of undergoing isotype switching in vivo. Following immunization, polyclonal antisera may be produced from such a transgenic animal. The transgenic non-human animals produced by the method of Surani et al. are able to produce, in one embodiment, IgG, IgA, and/or IgE antibodies that are encoded by human immunoglobulin genetic sequences and which also bind specific human antigens with high affinity.

DeBoer et al. (U.S. Pat. No. 5,633,076) and Meade et al. (U.S. Pat. No. 5,849,992) both disclose the production of transgenic cows which produce antibodies in their milk. DeBoer et al. produce transgenic cows by introducing a transgene, encoding an antibody gene operably linked to a mammary specific promoter, into a cow zygote. Meade et al. produce transgenic mammals which express antibodies in their milk by introducing downstream of a mammary specific promoter foreign DNA segments encoding specific paired immunoglobulin heavy and light chains.

However, the use of transgenics to produce domestic animals that express human antibodies for passive immunotherapy requires the solution of a number of problems. These include the levels at which human antibody transgenes might be expressed in non-human hosts, their ability to undergo class switching, affinity maturation and the immunogenicity in humans of inappropriately glycosylated human antibody. These problems stem from the introduction and expression of human antibody genes in non-human cells. A system that would allow for the introduction of human hematopoietic stem cells into non-humans, especially large animals of agricultural interest such as bovines and other ungulates (e.g., cattle, sheep, or goats), and their development into immunocompetent human B Cells would provide a comprehensive solution of these problems.

However, the immune system poses a major barrier to the introduction of foreign hematopoietic stem cells into an animal of another species. With respect to this barrier, it has been reported that the immune system can potentially be disabled by targeted disruption of rag-1 or rag-2 (recombinase activating gene) (hereinafter rag-1 knockout or rag-2 knockout). (See e.g., Martin et al., J Clin. Endocrinol. 79(3): 716-723 (1994); Mazurier et al., J. Interferon Cyylokline Res. 19(5): 533-541 (1999); Goldman et al., Br. J. Haematol. 103(2): 335-342 (1998)). Also, the production of IgM knockout mice that do not express functional endogenous B-cells have been reported. (See Ehrenstein et al., Proc. Natl. Acad. Sci., USA 95(17): 10089-10093 (1998); and Erlandsson et al., Eur. J. Immunol. 28(8): 2355-2365 (1998)). Rag-1 or rag-2 knockout animals potentially are unable to conduct the gene rearrangements that are necessary to generate the antigen receptors of B or T lymphocytes. Consequently, they do not develop native B or T cells. Moreover, because these animals do not produce B and T lymphocytes, the use of rag-1 or rag-2 knockout mice for engraftment of human hematopoietic stem cells has been reported.

Particularly, such a system has been developed in mice, wherein human hematopoietic progenitor cells have been added to rag-2 knockout mice. Yahata et al., Immunol. Lett. 62(3): 165-170 (1998) discloses transferring IL-12-induced splenic hematopoietic progenitor cells into rag-2 knockout mice to reconstitute their immune system. This resulted in the production of mice having stably engrafted therein both human B and T lymphocytes. However, while the development of human B and T lymphocytes in mice has been reported, there has been no report of human or other heterologous species hematopoietic stem cells stably engrafted into an ungulate or any indication that such cells, if stably engrafted will begin to develop into fully immunocompetent B and T cells when implanted into ungulates that do not produce B cells because of a genetic modification, e.g., IgM, Igα, EIA, BSAP, EBF, rag-1, or rag-2 knockout animals other than mice, and more specifically large agricultural animals such as cattle and other ungulates.

While it is anticipated that ungulates will be able to become stably engrafted with human stem cells and provide for the development of xenogeneic immunocompetent B and T cells in ungulates and other hoofed animals for which endogenous antibody production has been knocked out, e.g., by knockout of IgM, rag-1 or rag-2 gene, this outcome may not be feasible for various reasons. For example, natural killer cells do not depend on the rearrangement of antigen receptor genes for their cell killing activities. Consequently foreign lymphocytes, e.g., human lymphocytes potentially may be attacked by endogenous natural killer cells and thereby prevent the establishment of human B and T cells populations in B cell deficient ungulates, e.g., IgM, rag-1 or rag-2 deficient animals (provide for stable engraftment). Furthermore, the manner by which B cells and antibodies develop in humans is quite different from, for example, cattle or other ungulates. In humans, B cells arise in bone marrow and the primary repertoire is diversified by extensive rearrangement and junctional diversity. By contrast, in cattle, bone marrow is not the site of B cell origin. Primary repertoire diversification takes place in the spleen and gut associated lymphoid tissue rather than in bone marrow. Also, repertoire diversification in cattle uses relatively few rearrangements and little junctional diversity. Most of the diversity seen in the primary repertoire is the result of massive, variable region focused somatic mutation of rearranged genes. The sharp differences in B cell development and primary repertoire development between humans and cattle makes it unpredictable whether a functional and diverse repertoire of human B cells will develop from human hematopoietic stem cells transplanted into cattle and other ungulates and hoofed animals.

Furthermore, until now, various technical barriers have prevented the creation of ungulates, and other large agricultural animals, e.g., cattle, sheep, horses, goats, buffalo, that have been genetically manipulated in order to knockout antibody production, e.g. by genetically knocking out B cell production and optionally T cell production. Particularly, the use of conventional protocols for obtaining double knockouts in primary cell lines with limited life spans in culture is uncertain and difficult. The present inventor propose a method that should overcome these barriers and provides a protocol for producing ungulates having a double knockout that prevents B cell formation, e.g., E2A, EBF, BSAP, IgM, rag-1 and rag-2 knockout ungulates, especially cattle which have stably engrafted foreign B and T lymphocytes, preferably human, canine, feline, rat or murine, and which produce foreign immunoglobulins in their serum of the species of origin of the particular engrafted hematopoietic stem cells.

### SUMMARY OF THE INVENTION

A major object of the present invention is to provide a method for producing a cloned ungulate wherein the expression of both copies of a gene essential for B cell formation, e.g., Igα, IgM, E1A, EBF, BSAP, rag-1 or rag-2 gene have been eliminated, which said method comprises:

(i) producing an ungulate cell wherein the expression of both copies of a gene which is essential for antibody or B cell production, e.g., Igα, IgM (mμ) EBF, E2A, BSAP, rag-1 and/or rag-2 gene is eliminated by targeted disruption;

(ii) using said cell or nucleus thereof as a donor cell for nuclear transfer by fusing or inserting such donor cell or nucleus with a suitable recipient cell, e.g., an enucleated oocyte or blastomere and activating the resulting nuclear transfer unit and/or the oocyte prior to or simultaneous to nuclear transfer and culturing in a suitable medium to produce a nuclear transfer embryo;

(iii) introducing said nuclear transfer embryo into a female surrogate; and

(iv) obtaining a cloned ungulate that expresses the genotype of the donor differentiated cell, in which expression of both copies of the IgM (mu), Igα, E2A, EBF, BSAP, rag-1 gene and/or rag-2 gene has been knocked out.

Another object of the invention is to produce ungulates, or other hoofed animals, preferably cattle, wherein endogenous antibody production is knocked out non-genetically, i.e., by the administration of a monoclonal antibody against endogenous IgM which is administered while the animal is in utero, and engrafting heterologous hematopoietic stem cells, preferably human, canine, murine or feline in utero or shortly after birth.

Still another object of the invention involves the combination of genetic and non-genetic approaches in order to obtain cattle or other ungulates which produce human immunoglobulins or that of other species in their serum by producing an animal that contains and expresses a chromosomal minilocus containing genes necessary for non-ungulate antibody production, e.g., human antibody production, and by administering to such animal while in utero an antibody produced against endogenous bovine antibody so as to ablate B cells that express endogenous bovine antibodies and selectively retain B cells that produce non-bovine antibodies.

A further object of the present invention is to provide a method for producing a ungulate cell, preferably bovine wherein the expression of both copies of the Igα, IgM heavy chain (mu) rag-1, rag-2, EBF, E2A, or BSAP gene have been eliminated by targeted disruption, said method comprising the following steps:

(a) contacting a desired ungulate cell, preferably a differentiated cell, with at least one DNA construct which upon interaction with at least one of the Igα, IgM heavy chain gene, rag-1, rag-2, EBF, E2A, or BSAP gene is capable of eliminating the expression by targeted disruption of one copy of said gene;

(b) using said ungulate cell or the nucleus thereof as a nuclear transfer donor to produce a nuclear transfer embryo wherein one or both copies of such gene have been knocked out;.

(c) implementing said nuclear transfer embryo into an animal to produce a fetus and obtaining a cell, preferably a differentiated somatic cell is from such embryo, and contacting same with a second DNA construct that eliminates the expression of the second copy of the same gene, i.e., Igα, IgM, rag-1, rag-2, EBF, E2A, or BSAP by homologous recombination;

(d) using the resulting double knockout cell is used as a nuclear transfer donor to produce a second nuclear transfer embryo which is implanted into an ungulate and producing a fetus or offspring wherein both copies of said gene are knocked out and which animal does not produce functional B cells.

It is a further object of the present invention to provide a method for producing a cloned ungulate wherein the expression of both copies of the Igα, IgM heavy chain, E2A, EBF, BSAP, rag-1 and/or rag-2 genes have been eliminated, wherein said method comprises:

(i) producing an ungulate cell wherein the expression of both copies of the Igα, IgM heavy chain, rag-1, rag-2, EBF, E2A, or BSAP gene have been eliminated;

(ii) using said cell as a donor cell for nuclear transfer by introducing said cell or DNA derived therefrom into a suitable recipient cell, preferably in metaphase II, and most preferably an enucleated metaphase II oocyte or blastomere;

(iii) fusing said donor cell or nucleus and recipient cell, activating the resulting nuclear transfer unit or recipient cell, during and/or after fusion, and culturing in a suitable culture medium to produce a nuclear transfer embryo;

(iv) introducing said nuclear transfer embryo into a female surrogate;

(v) obtaining a cloned ungulate that expresses the genotype of the donor cells in wherein both copies of the Igα, IgM heavy chain, rag-1, rag-2, EBF, E2A, or BSAP genes have been eliminated;

(vi) optionally introducing into the cloned ungulate xenogeneic hematopoietic stem cells, preferably human, canine, feline, or murine hematopoietic stem cells.

It is a related object of the invention to collect B cells from said animal.

It is yet another object of the present invention to isolate polyclonal or monoclonal xenogeneic antibodies from cloned ungulates preferably human, canine, feline or murine antibodies wherein both copies of the Igα, IgM heavy chain, rag-1, rag-2, EBF, E2A, or BSAP genes have been eliminated.

It is yet another object of the present invention to produce antigen specific polyclonal or monoclonal xenogeneic antibodies, preferably human, canine, feline or murine by immunization of cloned ungulates wherein both copies of the Igα, IgM heavy chain, rag-1, rag-2, EBF, E2A, or BSAP genes have been eliminated with xenogeneic hematopoietic stem cells of a different species.

It is another object of the invention to provide cloned ungulates wherein both copies of the Igα, IgM, rag-1, rag-2, EBF, E2A, or BSAP gene have been knocked out by

(1) producing a female ungulate cell wherein one copy of the Igα, IgM, rag-1, rag-2, EBF, E2A, or BSAP has been knocked out by homologous recombination;

(2) producing a male ungulate cell line wherein one copy of the Igα, IgM, rag-1, rag-2, EBF, E2A, or BSAP has been knocked out by homologous recombination;

(3) using a female and male cell produced according to (1) and (2) as a nuclear transfer donors to respectively produce a cloned female and male ungulate, each respectively having one copy of the Igα, IgM, rag-1, rag-2 , EBF, E2A, or BSAP gene knocked out;

(4) mating said male and female knockout animals and selecting for progeny wherein both copies of a gene essential for B cell production have been knocked out by homologous recombination, e.g., the Igα, IgM, rag-1, rag-2, EBF, E2A, or BSAP; and optionally;

(5) introducing xenogeneic, preferably human, canine, feline or murine hematopoietic stem cells into said cloned ungulate.

### BRIEF DESCRIPTION OF THE DRAWINGS

FIG. 1. This figure contains a schematic of a targeting construct used for effecting inactivation of the rag-2 gene. In the Figure: the organization of the endogenous rag-2 gene is shown with an arrow representing the direction of transcription; and the targeting construct maintains the sequences 5′ and 3′ of the rag-2 coding region and the coding region is disrupted with a neomycin gene in the opposite transcriptional orientation.

FIG. 2. This figure contains the sequence of the bovine rag-2 gene.

### DETAILED DESCRIPTION OF THE PREFERRED EMBODIMENTS OF THE INVENTION

The present invention relates to the production of xenogeneic antibodies, preferably human, canine, feline or murine antibodies in large agricultural animals, i.e., ungulates, and other large hoofed animals such as bovines, pigs, horses, sheep, buffalo and goats. As noted previously, the immune system poses a major barrier to the introduction of xenogeneic hematopoietic stem cells such as those of human origin into non-human animals. The present inventors remove this barrier in cattle by targeted disruption of both copies of at least one gene which is essential for functional B cells, preferably IgM heavy chain, Igα, EBF (a transcription factor essential for B cell development(O'Riordan et al., Immunity 11: 21-31 (1999));. E2A (another transcription factor essential for B cell development) (Bain et al., Cell 79: 885-892 (1994)), and BSAP (still another transcription factor essential for B cell development (Urbanek et al., Cell 79: 901-912 (1994)). For example, in the case of rag knockout animals, they are unable to conduct the gene rearrangements that are necessary to generate the antigen receptors of B or T lymphocytes. Consequently, they do not develop endogenous B or T lymphocytes. Because they will not produce endogenous B and T lymphocytes, these rag-1 or rag-2 knockout cattle should not reject human or other species hematopoietic stem cells, and human B cells that develop from them should proceed by mechanisms that employ antibody or cytotoxic T cells. The development of human T cells and human immunoglobulins should also proceed in these animals.

More specifically, the present invention provides a method for producing xenogeneic, preferably human antibodies in a cloned animal, such as an ungulate, which comprises producing a cloned non-human animal which has been genetically modified to delete or inactivate both copies of at least one gene essential for B cell production, e.g., Igα, IgM (mu), BSAP, E2A, EBF, rag-1 or rag-2 gene. These cloned non-human animals are engrafted in utero or shortly after birth with xenogeneic hematopoietic stem cells, e.g., human, canine, feline, or murine stem cells such as mouse, or rat. Most preferably human hematopoietic stem cell-enriched preparations obtained from human umbilical cord or peripheral blood are used for engraftment. After such administration, these cloned animals ideally will comprise xenogeneic human B and T lymphocytes stably engrafted and will not produce endogenous B cells.

When responding to immunogenic antigens naturally encountered by the non-human host or when specifically immunized, these engineered animals will make xenogeneic, preferably human antibodies in xenogeneic, preferably human B lineage cells. Large amounts of antibody will be produced because there will be complete compatibility between human antibody genes and the intracellular factors that regulate their expression. The antibodies produced should have the post-translational modifications (glycosylation patterns, etc.) that are typical of human antibodies made in human systems. Immune responses should be efficient because the T cell help will be provided by compatible T cells, e.g., human T cells. Furthermore, a variety of useful classes of xenogeneic, preferably human antibodies of high affinity can be produced because the intracellular factors that regulate switching and somatic mutation-driven affinity maturation are compatible with the xenogeneic, preferably human antibody genes. The presence of compatible T cells should enable and facilitate antibody class switching and the somatic hypermutation of rearranged antibody genes.

Therefor, in one embodiment, the present invention involves producing a cloned genetically engineered or transgenic ungulate, in which the expression of both copies of a desired gene essential for B cell production, e.g., Igα, EBF, E2A, or BSAP, the IgM, rag-1 or rag-2 gene has been knocked out. This is effected by genetically modifying a cell obtained from such animal in vitro, using an appropriate targeting construct, and using the resulting genetically modified cell or nucleus, as a nuclear donor for nuclear transfer by fusing or inserting such cell or nucleus into a suitable recipient cell, e.g. a cell in metaphase II, preferably an oocyte or blastomere. Suitable genetically modified cells include germ cells, embryonic cells, and differentiated (somatic) cells, and most preferably will comprise differentiated cells. Differentiated ungulate cells according to the present invention are those cells which are past the early embryonic disc stage (in the case of bovines corresponds to day 10 of bovine embryogenesis). Suitable differentiated cells may be derived from ectoderm, mesoderm or endoderm.

Suitable donor cells may be obtained by known methods. Examples of differentiated donor cells useful in the present invention include, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the donor cells used for nuclear transfer may be obtained from different organs, e.g., skin, lung, pancreas, liver, stomach, intestine, heart, reproductive organs, bladder, kidney, urethra and other urinary organs, etc. These are just examples of suitable donor cells. Suitable donor cells, i.e., cells useful in the subject invention, may be obtained from any cell or organ of the body. This includes all somatic or germ cells, and also includes embryonic stem and germ cells, e.g. primordial germ cells.

Standard protocols for constructing knockout animals are provided, for example, in Thomas, K. R. et al., “High frequency targeting of genes to specific sites in the mammalian genome,” Cell 44: 419-428 (1986); Thomas, K. R. et al., “Site-directed mutagenesis by targeting in mouse embryo-derived stem cells,” Cell 51: 503-512 (1987); and Mansour, S. L. et al., “Disruption of the proto-oncogene int-2 in mouse embryo-derived stem cells: a general strategy for targeting mutations to non-selectable genes,” Nature 336: 348-352 (1988). As noted previously, obtaining a double knockout in primary cell lines with limited life spans in culture is difficult and uncertain. The present inventors have solved this problem in ungulates by modifying these standard protocols.

Preferably, fibroblast cells, most preferably fetal fibroblasts, will be genetically modified to obtain an ungulate cell which is homozygous for a gene essential for B cell production, e.g., Igα, E2A, EBF, BSAP, IgM, a rag-1 or rag-2 deletion. Fibroblast cells are an ideal cell type because they can be obtained from developing fetuses and adult animals in large quantities. Fibroblast cells have recently been reported to be well suited for use in cloning procedures. Of importance herein, these cells can be easily propagated in vitro with a rapid doubling time and can be clonally propagated permitting their use in gene targeting procedures.

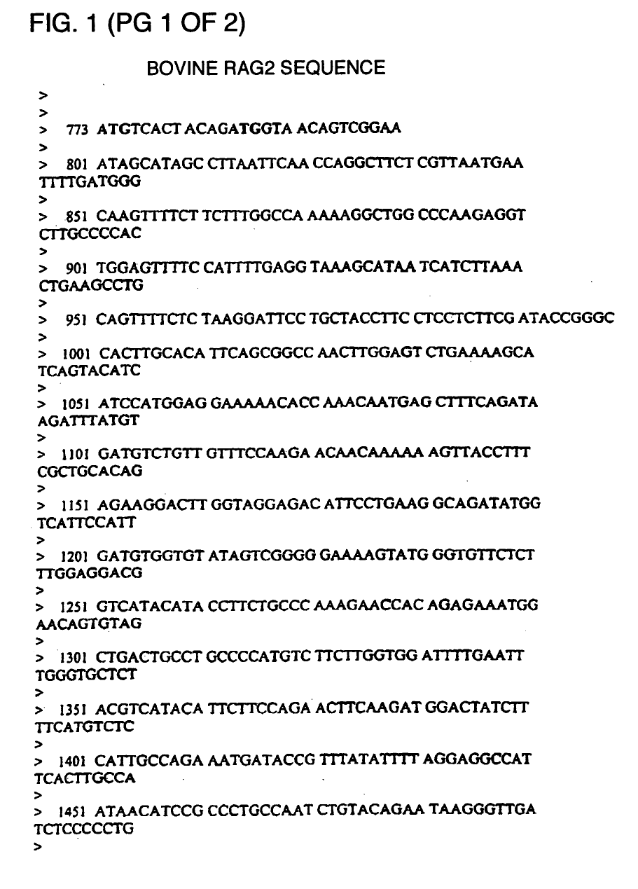

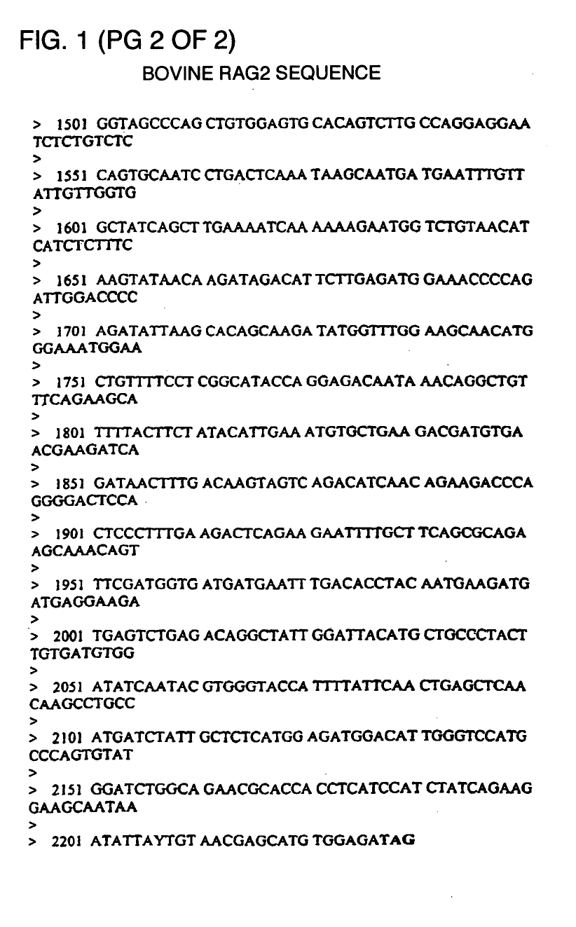

In the present invention fibroblast cells or other suitable non-cells obtained from a particular ungulate, e.g., a bovine, are contacted, e.g. by transfection with a first vector construct that is designed such that it homologously recombines with one copy of a gene essential for B cell production, and resulting in the inactivation thereof. Typically, the targeting construct will comprise portions of the targeted gene, an intervening sequence that is inserted in place of the target gene, and at least one marker gene that provides for selection of homologous recombinants. The DNA construct is introduced into the cell by known means, e.g. transfection, microinjection, electroporation, and transformation. Thus, in the invention the DNA of a desired ungulate cell, e.g., a bovine fibroblast, is contacted with a DNA construct that homologously recombines a gene involved in B cell production with the bovine genome and results in the targeted deletion or inactivation of one copy of the target gene, e.g., IgM, Igα, rag-1, rag-2, EBF, E2A or BSAP. An exemplary targeting construct for effecting deletion of the rag-2 gene is depicted in FIG. 1. Methods for constructing vectors and the use thereof for effecting targeted deletion by homologous recombination are the subject of numerous patents which are incorporated by reference herein. These patents include e.g., U.S. Pat. Nos. 6,143,566;0 6,139,835; 6,074,853; 6,010,908; 5,998,144; 5,981,214; 5,945,334; 5,925,544; 5,783,385; 5,731,411; 5,721,367; 5,776,744; 5,614,396; 5,574,2_{—}5; 5,527,674; 5,204,244; and 5,468,629.

Successfully genetically modified cells, preferably fibroblasts, or DNA therefrom which are hemizygous for the target gene, e.g., Igα, E2A, EBF, BSAP, IgM, rag-1 or rag-2 gene, are then inserted or fused with suitable recipient cells, preferably enucleated oocytes or blastomere, using standard nuclear transfer techniques. The resulting nuclear transfer units are then allowed to develop, preferably until about the 40 day gestation state or later, at which point donor cells are obtained therefrom, e.g., fetal fibroblast cells and these cells are subject to a second round of gene targeting. The second vector construct, typically comprises the same DNA sequences as the first vector construct except that it comprises a different selective marker than used in the first construct. This vector is introduced into donor cells, e.g., fetal fibroblast cells again by known methods, e.g., transfection. Double knockout cells, e.g., fibroblast cells or cell nucleus are obtained are then fused or inserted into suitable recipient cells, preferably enucleated oocytes, again using standard nuclear transfer techniques known in the art. The resulting embryos are allowed to develop fully, in utero. Isolation of double knockout cells can be confirmed e.g. by known detection methods, e.g. DCR.

Alternatively, male and female cell lines are obtained wherein one copy of a gene essential for B cell production is knocked out or inactivated, e.g., EBF, E2A, BSAP, Igα, IgM, rag-1 or rag-2 as described, these male and female cell lines or DNA therefrom are each used as donor cells or nuclei for nuclear transfer to respectively produce a cloned female and male animal that comprises one copy of the IgM, rag-1 or rag-2 gene knocked out, or inactivated, the cloned animals are mated, and progeny are selected wherein both copies of the targeted gene, e.g., E2A, Igα, EBF, BSAP, IgM, rag-1 or rag-2 gene have been knocked out or inactivated. Again cells that are knockout can be confirmed by DCR detection methods.

In the present invention, suitable ungulate and hooved animals include by way of example sheep, cows, pigs, horses, guar, antelope, caribou, deer, goats, buffalo, etc. Methods for obtaining oocytes from such animals suitable for use in nuclear transfer are well known in the art. Preferably, large ungulates, and most preferably bovines will be cloned.

Additionally, nuclear transfer techniques or nuclear transplantation techniques are also known in the literature. See, in particular, Campbell et al., Theriogenology 43: 181 (1995); Collas et al., Mol. Report Dev. 38: 264-267 (1994); Keefer et al., Biol. Reprod. 50: 935-939 (1994); Sims et al., Proc. Natl. Acad. Sci., USA 90: 6143-6147 (1993); WO 94/26884; WO 94/24274, and WO 90/03432, which are incorporated by reference in their entirety herein. Also U.S. Pat. Nos. 4,944,384 and 5,057,420 described procedures for bovine nuclear transplantation.

A particularly preferred method is disclosed in recently issued U.S. Pat. No. 5,945,577, the contents of which are incorporated by reference herein. This patent contains claims directed to the use of proliferating somatic cells or nuclei as donors for nuclear transfer. Alternatively, quiescent donor cells or nuclei therefrom can be used as donors for nuclear transfer as discussed by Ian Wilmut and Keith Campbell in WO 09707668A, WO 09707669A1, WO 00018902A1 and WO 00022098A1, all of which are incorporated by reference in their entirety herein.

As noted, methods for isolation of oocytes suitable for use as recipient cells in nuclear transfer are also well known in the art. Typically, this will comprise isolating oocytes from the ovaries or reproductive tract of an ungulate or other hooved mammal, e.g., a bovine. A readily available source of bovine oocytes is slaughterhouse materials

For the successful use of techniques such as genetic engineering, nuclear transfer and cloning, oocytes are generally matured in vitro before these cells are used as recipient cells for nuclear transfer. This process generally requires collecting immature (prophase I) oocytes from suitable, e.g., ungulate ovaries, specifically bovine ovaries obtained at a slaughterhouse, and maturing the oocytes in a maturation medium prior to fertilization or enucleation until the oocyte attains the metaphase II stage, which in the case of bovine oocytes generally occurs about 18-24 hours post-aspiration. For purposes of the present invention, this period of time is known as the “maturation period.” As used herein for calculation of time periods, “aspiration” refers to aspiration of the immature oocyte from ovarian follicles.

Alternatively, metaphase II stage oocytes, which are matured in vivo can be used for nuclear transfer. For example, mature metaphase II oocytes are collected surgically from either non-superovulated or superovulated cows or heifers 35 to 48 hours past the onset of estrus or past the injection of human chorionic gonadotropin (hCG) or similar hormone.

The stage of maturation of the oocyte at enucleation and nuclear transfer can affect the success of NT methods. (See e.g., Prather et al., Differentiation, 48: 1-8, 1991). In general, successful mammalian embryo cloning practices use the metaphase II stage oocytes as the recipient cell because at this stage it is believed that the oocyte can be or is sufficiently “activated” to treat the introduced nucleus as it does a fertilizing sperm. In domestic animals, and especially cattle, the oocyte activation period generally ranges from about 16-52 hours, preferably about 2842 hours post-aspiration. However this may vary somewhat across different species. One skilled in the art can determine an appropriate stage of maturation For example, immature oocytes may be washed in buffered hamster embryo culture medium (HECM) as described in Seshagine et al., Biol. Reprod. 40: 544-606, 1989, and then placed into drops of maturation medium consisting of 50 microliters of tissue culture medium (TCM) 199 containing 10% fetal calf serum which contains appropriate gonadotropins such as luteinizing hormone (LH) and follicle stimulating hormone (FSH), and estradiol under a layer of lightweight paraffin or silicon at 39° C.

After a fixed time maturation period, which ranges from about 10 to 40 hours, and preferably about 16-18 hours, the oocytes are in the case of bovine oocytes typically enucleated. Prior to enucleation the oocytes are preferably removed and placed in HECM containing 1 milligram per milliliter of hyaluronidase prior to removal of cumulus cells. This may be effected by repeated pipetting through very fine bore pipettes or by vortexing briefly. The stripped oocytes are then screened for polar bodies, and the selected metaphase II oocytes, as determined by the presence of polar bodies, are then used for nuclear transfer. Enucleation follows.

Enucleation may be effected by known methods, such as described in U.S. Pat. No. 4,994,384 which is incorporated by reference herein. For example, metaphase II oocytes are either placed in HECM, optionally containing 7.5 micrograms per milliliter cytochalasin B, for immediate enucleation, or may be placed in a suitable medium, for example an embryo culture medium such as CR1aa, plus 10% estrus cow serum, and then enucleated later, preferably not more than 24 hours later, and more preferably 16-18 hours later.

Enucleation may be accomplished microsurgically using a micropipette to remove the polar body and the adjacent cytoplasm. The oocytes may then be screened to identify those of which have been successfully enucleated. This screening may be effected by staining the oocytes with 1 microgram per milliliter 33342 Hoechst dye in HECM, and then viewing the oocytes under ultraviolet irradiation for less than 10 seconds. The oocytes that have been successfully enucleated can then be placed in a suitable culture medium.

A single ungulate cell or that of another hooved animal, preferably one that produces a large amount of blood, of the same or different species as the enucleated oocyte or a nucleus thereof will then be transferred into the perivitelline space of the enucleated oocyte used to produce the NT unit. The donor cell and the recipient cell, i.e., enucleated oocyte will be used to produce NT units according to methods known in the art. For example, the cells may be fused by electrofusion. Electrofusion is accomplished by providing a pulse of electricity that is sufficient to cause a transient breakdown of the plasma membrane. This breakdown of the plasma membrane is very short because the membrane reforms rapidly. Thus, if two adjacent membranes are induced to breakdown and upon reformation the lipid bilayers intermingle, small channels will open between the two cells. Due to the thermodynamic instability of such a small opening, it enlarges until the two cells become one. Reference is made to U.S. Pat. No. 4,997,384 by Prather et al., (incorporated by reference in its entirety herein) for a further discussion of this process. A variety of electrofusion media can be used including e.g., sucrose, mannitol, sorbitol and phosphate buffered solution. Fusion can also be accomplished using Sendai virus as a fusogenic agent (Graham, Wister Inot. Symp. Monogr. 9: 19 (1969)).

In some cases (e.g. with small donor nuclei) it may be preferable to inject the nucleus directly into the oocyte rather than using electroporation fusion. Such techniques are disclosed in Collas and Barnes, Mol. Reprod. Dev. 38: 264-267 (1994), incorporated by reference in its entirety herein.

The NT unit may be activated by known methods. Such methods include, e.g., culturing the NT unit at sub-physiological temperature, in essence by applying a cold, or actually cool temperature shock to the NT unit. This may be most conveniently done by culturing the NT unit at room temperature, which is cold relative to the physiological temperature conditions to which embryos are normally exposed.

Alternatively, activation may be achieved by application of known activation agents. For example, penetration of oocytes by sperm during fertilization has been shown to activate prefusion oocytes to yield greater numbers of viable pregnancies and multiple genetically identical calves after nuclear transfer. Also, treatments such as electrical and chemical shock may be used to activate NT embryos after fusion. Suitable oocyte activation methods are the subject of U.S. Pat. No. 5,496,720, to Susko-Parrish et al., herein incorporated by reference in its entirety.

Additionally, activation may be effected by simultaneously or sequentially:
increasing levels of divalent cations in the oocyte, and
reducing phosphorylation of cellular proteins in the oocyte.

This will generally be effected by introducing divalent cations into the oocyte cytoplasm, e.g., magnesium, strontium, barium or calcium, e.g., in the form of an ionophore. Other methods of increasing divalent cation levels include the use of electric shock, treatment with ethanol and treatment with caged chelators.

Phosphorylation may be reduced by known methods, e.g., by the addition of kinase inhibitors, e.g., serine-threonine kinase inhibitors, such as 6-dimethylaminopurine, staurosporine, 2-aminopurine, and sphingosine.

Alternatively, phosphorylation of cellular proteins may be inhibited by introduction of a phosphatase into the oocyte, e.g., phosphatase 2A and phosphatase 2B.

A preferred protocol procedure involves the use of cycloheximide and cytochalasin D and the media described below. It shall be noted that this is exemplary of suitable activation methods and media, and is not essential to the invention:

### Preparation of Activation, and Culture Plate

An activation plate is commenced by combining 500 ul of ACM media (described below), 2.5 ul CHX, 0.5 ul Cytochalasin D, on a tissue culture plate, and by placement of activation media in 35 ul micro drops which are treated with mineral oil, just until the tops of the drops become covered.

Thereafter, a 1% FCS culture plate for day 0 to day 4 old embryos is prepared by combining 500 ul ACM plus 5 ul FCS. This is again effective using tissue plates prepared using 35 ml which are cover micro drops of 35 ul with oil. The activation and culture plates are then equilibrated for a minimum of 2 hours before transferring the oocytes or embryos to another plate.

### Preparing Oocytes for Activation

After oocytes have matured (at least 20 hours) they are stripped of their cumulus cells to facilitate activation. This is effected by use of a solution of hyaluronidase and TLHepes in an amount appropriate to effect activation. Two ml of the activate solution are aliquoted into a 3 5 mm petri dish to rinse oocytes after removal from maturation media. Another 2 ml is used for stripping and is placed in a 15 ml conical tube. Typically, up to 200-300 oocytes may be stripped in two volume of media.

Oocytes are then removed from maturation media while collecting as little fluid as possible and are transformed to a hyaluronidase rinse plate. Oocytes allowed to soak for approximately 2-3 minutes, with the swirling plate often in order to dilute the maturation media and rinse oocytes. Oocytes are removed from rinse plate and placed in 15 ml conical for vortexing. Vortexing is used to strip oocytes, e.g., for about 5-6 minutes at a medium speed (Fisher Vortex-Genie 2).

After vortexing oocytes are placed on a 35 mm petri plate and rinsed in a 15 ml tube using 2 ml TLHepes also placed in the same dish. Oocytes are retrieved and rinsed using 2 TLHepes. If the oocytes are younger than 24 hours when stripped, they preferably are placed into equilibrated ACM and held in an incubator until at lest about 24 hours old.

### Ionomycin Treatments and Subsequent Rinses

Oocytes preferably are approximately 24-30 hours old upon activation. Activation is preferably effected by use of a 2 ml solution of Z- 1 media and ionomycin which is allowed to warm on a heating stage, under an opaque cover to eliminate light, for about 2-3 min. The media is then heated to approximately 38° C., and oocytes to be activated are transferred into ionomycin solution for about 4 minutes. After about 4 minutes has elapsed oocytes are removed from media and immediately place in TLHepes to rinse. After about 3-4 rinsers, oocytes are transferred to an equilibrated activation plate and incubated for about 6 hours.

### Activation Plate Incubation and Rinses to Culture Plates

After incubation period is completed oocytes are removed from activation plates and again rinsed, preferably about 4 times in TLHepes. After rinses are completed, the oocytes are transferred into ACM+1% FCS culture plates, and then incubated until day 4 (activation date=d0).

On day 4, 4 culture plates are prepared by combining 500 ul ACM and 50 ul FCS. After thorough mixing the media is placed as micro drops (35 ul) onto a tissue culture plate, which again is covered in mineral oil and incubated preferably for a minimum of about 2 hours to equilibrate. The oocytes are transferred directly from the first culture plate on the second (ACM+10% FCS), and oocytes/embryos are then counted. The cleavage rate is calculated by taking the number of embryos cleaved and dividing by the number of oocytes initially activated. At days 7, and 8, embryos are observed for blastocyst formation and additional embryo that contain blastocoel are counted. The blastocyst rate is obtained by dividing the number of blastocysts by the number of oocytes originally activated, to obtain the blastocyst rate.

Media and Formulations Used in Above Described Activation Procedures

ACM Media

| | | |
|---|---|---|
| NaCl | 0.580 | g |
| NaHCO₃ | 0.209 | g |
| KCl | 0.022 | g |
| L-glutamine | 0.015 | g |
| *CaCl₂2H₂0 | 0.004 | g |
| Pyruvic Acid | 2 | ml |
| BME | 2 | ml |
| MEM | 1 | ml |
| Pen/Strep | 1 | ml |
| Lactic Acid | 14 | ul |
| Phenol Red | 100 | ul |
| BSA (fatty acid free) | 0.300 | g |

Z-1 Media

| | | |
|---|---|---|
| H20 | 500 | ml |
| NaCl | 3.300 | g |
| KCl | 0.120 | g |
| NaHCO₃ | 0.084 | g |
| NaH₂Po₄H₂O | 0.024 | g |
| *CaCl₂2H₂0 | 0.150 | g |
| *MgCl₂6H₂0 | 0.050 | g |
| Hepes | 1.200 | g |
| Pen/Strep | 5 | ml |
| Lactic Acid | 930 | ul |
| Phenol Red | 500 | ul |
| BSA (fatty acid free) | 0.500 | g |

TLHepes

| | | |
|---|---|---|
| I120 | 500 | ml |
| NaCl | 3.300 | g |
| KCl | 0.120 | g |
| NaIICO₃ | 0.084 | g |
| NaH₂Po₄H₂O | 0.024 | g |
| *CaCl₂2H₂0 | 0.150 | g |
| *MgCl₂6H₂0 | 0.050 | g |
| Hepes | 1.200 | g |
| Pen/Strep | 5 | ml |
| Lactic Acid | 930 | ul |
| Phenol Red | 500 | ul |
| BSA (fatty acid free) | 0.500 | g |

Ionomycin Activation Media

| | | |
|---|---|---|
| Z-1 Media | 2 | ml |
| Ionomycin | 2 | ul |

Hyluronidase Solution for Stripping Oocytes

1 ml TLHepes/1 mg Hyluronidase

Activation Media Plates

| | | |
|---|---|---|
| ACM | 500 | ul |
| Cycloheximide | 2.5 | ul |
| Cytochalasin D | .5 | ul |

Culture Plate d0-d4

| | | |
|---|---|---|
| ACM | 500 | ul |
| FCS | 5 | ul |

Culture Plate d4 d8

| | | |
|---|---|---|
| ACM | 500 | ul |
| FCS | 5 | ul |

Activated NT units can be cultured in a suitable in vitro culture medium until the generation of CICM cells and cell colonies. Culture media suitable for culturing and maturation of embryos are well known in the art. Examples of known media, which may be used for bovine embryo culture and maintenance, include Ham's F-10+10% fetal calf serum (FCS), Tissue Culture Medium-199 (TCM-199)+10% fetal calf serum, Tyrodes-Albumin-Lactate-Pyruvate (TALP), Dulbecco's Phosphate Buffered Saline (PBS), Eagle's and Whitten's media. One of the most common media used for the collection and maturation of oocytes is TCM-199, and 1 to 20% serum supplement including fetal calf serum, newborn serum, estrual cow serum, lamb serum or steer serum. A preferred maintenance medium includes TCM-199 with Earl salts, 10% fetal calf serum, 0.2 mM Na pyruvate and 50 μg/ml gentamicin sulphate. Any of the above may also involve co-culture with a variety of cell types such as granulosa cells, oviduct cells, BRL cells and uterine cells and STO cells.

Another maintenance medium is described in U.S. Pat. No. 5,096,822 to Rosenkrans, Jr. et al., which is incorporated herein by reference. This embryo medium, named CR1, contains the nutritional substances necessary to support an embryo.

Afterward, the cultured NT unit or units are preferably washed and then placed in a suitable media containing FCS well plates which preferably contain a suitable confluent feeder layer. Suitable feeder layers include, by way of example, fibroblasts and epithelial cells, e.g., fibroblasts and uterine epithelial cells derived from ungulates, chicken fibroblasts, murine (e.g., mouse or rat) fibroblasts, STO and SI-m220 feeder cell lines, and BRL cells.

The NT units are cultured on the feeder layer until the NT units reach a size suitable for transferring to a recipient female, or for obtaining cells which may be used to produce CICM cells or cell colonies. Preferably, these NT units will be cultured until at least about 2 to 400 cells, more preferably about 4 to 128 cells, and most preferably at least about 50 cells. Culturing is preferably effected under suitable conditions, i.e., about 38.5° C. and 5% C0₂, with the culture medium changed in order to optimize growth typically about every 2-5 days, preferably about every 3 days.

The methods for embryo transfer and recipient animal management utilized in the present invention are standard techniques for the embryo transfer industry. Synchronous transfers are advantageous to the success rate, i.e., in development of viable offspring after embryo transfer, i.e., the stage of the NT embryo is in synchrony with the estrus cycle of the recipient female. This advantage and how to maintain recipients are reviewed in Siedel, G. E., Jr. (“Critical review of embryo transfer procedures with cattle” in Fertilization and Embryonic Development in Vitro (1981), L. Mastroianni, Jr. and J. D. Biggers, ed., Plenum Press, New York, N.Y., page 323), the contents of which are hereby incorporated by reference. Preferably, activation and culturing is effected using cycloheximide and cytochalasin Dc8 described in the example.

According to the invention, ungulates which do not express endogenous antibodies, because of inactivation or knockout of a gene essential for B cell production, e.g., Igα, Igm (mu), E2A, EBF, BSAP, rag-1 or rag-2, will be injected in utero or shortly after birth, typically within about one week, and more preferably within the first 48 hours after birth, with xenogeneic hematopoietic stem cells. Methods for purifying such xenogeneic, preferably murine, canine, feline or human, or non-human primate hematopoietic stem cells are well known. Such methods typically use ligands that bind to stem cell markers. Such markers include CD34 and Thy-1. Known purification methods include flow cytometry, negative selection, immuno-purificatin, etc. For example, WO 99/23205 recently filed by Dick et al., discloses a method for producing purified human hematopoietic stem cells and is peripheral blood, and cord blood. Other methods are described in U.S. Pat. Nos. 5,763,197; 5,981,708; 5,763,266; and 5,914,108 incorporated by reference herein.

These animals are injected preferably with about 10⁷-10⁸ cells of a preparation of enriched hematopoietic stem cells, preferably human. It is anticipated that this will be sufficient to “reconstitute” the immune system of an ungulate, e.g., a cow, with xenogeneic (human) B and T cells. This may be effected via a single or multiple administration, e.g., if stable engraftment does not result after initial injection of stem cells. Also, higher cell numbers may be administered if necessary. Additionally, to facilitate engraftment of donor cells, cytokines or stromal cells may additionally be administered as this may facilitate the development of human or other stem cells into lymphoid lineages. This may be effected by administration of appropriate (homologous) hematopoietic cytokines, e.g., any of the interleukins, e.g., IL-1, IL-2, IL-3, IL-4, IL-5, IL-6, IL-7, IL-8, IL-9, IL-10, IL-11, IL-12, colony stimulating factors such as GM-CSF and others, e.g., erythropoietin. Alternatively, a gene encoding appropriate cytokines may be introduced during genetic modification of target cells. Alternatively or additionally, homologous bone marrow stromal cells may be introduced. These cytokines and stromal cells may be administered repeatedly before, simultaneously, or after stem cell infusion.

After the hematopoietic stem cells have been stably engrafted, the ungulates, e.g., bovine can be used to produce antibodies against desired antigens. These antigens include those to which the animal is naturally exposed, or antigens that are administered by exogenous means, e.g. by injection. Suitable antigens broadly include any antigen to which an antibody, e.g., human antibody, is desirably produced against. These antigens include by way of example antigens specific to infectious agents, such as viruses, bacteria, fungi, yeast, allergens, antigens expressed by tumor cells, disease markers, cytokines, signaling molecules, therapeutic agents, enzymes, cytokines, growth factors, lectins, among others.

After the stably engrafted animal, e.g., an IgM, rag-1, Igα, E2A, BSAP, EBF, rag-2 knockout ungulate has been exposed to factors, the antigen, the animal should elicit an immune response against such antigen resulting in the production of xenogeneic, e.g., human antibodies against such antigen. The serum from the animal, e.g., a bovine, which contains such antibodies can be used for effecting passive immunization against the antigen. Alternatively, the antibodies can be purified and isolated from the animal's serum by well known methods. These antibodies can be either monoclonal or polyclonal antibodies. Alternatively, the B cells can be isolated from the bovine and immortalized by fusing with, for example, myeloma cells, and the monoclonal antibodies secreted by these cells can be isolated using well known methods.

The following examples are presented in order to more fully illustrate the preferred embodiments of the invention. They should in no way be construed, however, as limiting the broad scope of the invention.

### EXAMPLE 1

The following procedures were used to generate bovine fibroblast cell lines in which one allele of the immunoglobulin heavy chain (mu) locus is disrupted by homologous recombination. A DNA construct for effecting IgM knockout was generated by the removal of introns 1-4 of the Mu locus which were replaced with a copy of neomycin resistance gene. Using this construct, neomycin resistant cell lines have been obtained which were successfully used in nuclear transfer procedures and blastocysts from these cell lines have been implanted into recipient cows. Additionally, some of these blastocysts were tested to confirm that targeted insertion into has occurred appropriately in the mu locus using PCR procedures. Blastocysts resulting from nuclear transfer procedures from several of the cell lines obtained indicated that heterozygous IgM-KO fetuses are in gestation. Additionally, both male and female cell lines that comprise a single IgM (mu) knockout have been produced. It is anticipated that mating of animals cloned from these cell lines will give rise to progeny wherein both copies of mu are inactivated. These procedures are discussed in greater detail below.

DNA Construct

The DNA used in all transfections described in this document was generated as follows:

The four main exons (excluding the transmembrane domain exons), CH1-4, are flanked by an XhoI restriction site at the downstream (CH4) end and an XbaI site at the upstream (CH1) end. The construct used for the transfection procedure consists of 1.8 kb of genomic sequence downstream of the XhoI site and 3.1 Kb of genomic sequence upstream of the XbaI site. A neomycin resistance marker was inserted between these two fragments on a 3.0 Kb fragment, replacing 2.4 Kb of DNA, originally containing CH1-4, from the originating genomic sequence. The backbone of the vector is pBluescriptII SK+(Stratagene) and the insert of 8.9 Kb was purified and used for transfection of bovine fetal fibroblasts. This construct is shown in FIG. 3.

Transfection /Knockout Procedures

Transfection of fetal bovine fibroblasts was performed using a commercial reagent Superfect Transfection Reagent (Qiagen, Valencia, Calif., USA), Catalog Number 301305.

Bovine fibroblasts were generated from disease-tested cattle at Hematech of Kansas/Cyagra of Kansas, sent to Hematech's Worcester Molecular Biology Labs and used for all experiments described.

The medium used for culture of bovine fetal fibroblasts consisted of the following components:
500 ml Alpha MEM (Bio-Whittaker # 12-169F)
50 ml fetal calf serum (Hy-Clone #A-1111-D)
2 ml antibiotic/antimyotic (Gibco/BRL #15245-012)
1.4 ml 2-mercaptoethanol (Gibco/BRL #21985-023)
5.0 ml L-Glutamine (Sigma Chemical #G-3126)
0.5 ml tyrosine tartrate (Sigma Chemical #T-6134)

On the day prior to transfection procedures, cells were seeded in 60 mm tissue culture dishes with a targeted confluency of 40-80% as determined by microscopic examination.

On the day of transfection, 5 ug of DNA, brought to a total volume of 150 ul in serum-free, antibiotic-free medium), was mixed with 20 ul of Superfect transfection reagent and allowed to sit at room temperature for 5-10 minutes for DNA-Superfect complex formation. While the complex formation was taking place, medium was removed from the 60 mm tissue culture dish, containing bovine fibroblasts to be transfected, and cells were rinsed once with 4 ml of phosphate-buffered saline. One milliliter of growth medium was added to the 170 ul DNA/Superfect mixture and immediately transferred to the cells in the 60 mm dish. Cells were incubated at 38.5 degrees Celsius, 55 carbon dioxide for 2.5 hours. After incubation of cells with the DNA/Superfect complexes, medium was aspirated off and cells were washed four times with 4 ml PBS. Five ml of complete medium were added and cultures were incubated overnight at 38.5 degrees C., 5% CO2. Cells were then washed once with PBS and incubated with one ml of 0.3% trypsin in PBS at 37° C. until cells were detached from the plate, as determined by microscopic observation. Cells from each 60 mm dish were split into 24 wells of a 24 well tissue culture plate (41.7 ul/well). One milliliter of tissue culture medium was added to each well and plates were allowed to incubate for 24 hours at 38.5 degrees C. and 5% CO2 for 24 hours.

During all transfection procedures, sham transfections were performed using a Superfect/PBS mixture containing no DNA, as none of those cells would be expected to contain the neomycin resistance gene and all cells would be expected to die after addition of G418 to the tissue culture medium. This served as a negative control for positive selection of cells that received DNA.

After the 24 hour incubation, one more milliliter of tissue culture medium containing 400 ug/ml G418 was added to each well, bringing the final G418 concentration to 200 ug/ml. Cells were placed back into the incubator for 7 days of G418 selection. During that period, both transfected and sham transfection plates were monitored for cell death and over 7 days, the vast majority of wells from the sham transfections contained few to no live cells while plates containing cells that received the DNA showed excellent cell growth.

After the 7 day selection period, the cells from wells at 90- 100% confluency were detached using 0.2 m 0.3% trypsin in PBS and were transferred to 35 mm tissue culture plates for expansion and incubated until they became at least 50% confluent, at which point, cells were trypsinized with 0.6 ml 0.3% trypsin in PBS. From each 35 mm tissue culture plate, 0.3 ml of the 0.6 ml cell suspension was transferred to a 12.5 cm2 tissue culture flask for further expansion. The remaining 0.3 ml was reseeded in 35 mm dishes and incubated until they attained a minimal confluency of approximately 50%, at which point cells from those plates were processed for extraction of DNA for PCR analysis. Flasks from each line were retained in the incubator until they had undergone these analyses and were either terminated if they did not contain the desired DNA integration or kept for future nuclear transfer and cryopreservation.

Screening for Targeted Integrations

As described above the DNA source for screening of transfectants containing the DNA construct was a 35 mm tissue culture dish containing a passage of cells to be analyzed. DNA was prepared as follows and is adapted from a procedure published by Laird et al. (Laird et al., “Simplified mammalian DNA isolation procedure”, Nucleic Acids Research, 19:4293). Briefly, DNA was prepared as follows:

A cell lysis buffer was prepared with the following components:
100 mM Tris-HCl buffer, pH 8.5
5 m EDTA, pH 8.0
0.2% sodium dodecyl sulfate
200 mM NaCl
100 ug/ml Proteinase K

Medium was aspirated from each 35 mm tissue culture dish and replaced with 0.6 ml of the above buffer. Dishes were placed back into the incubator for three hours, during which cell lysis and protein digestion were allowed to occur. Following this incubation, the lysate was transferred to a 1.5 ml microfuge tube and 0.6 ml of isopropanol was added to precipitate the DNA. Tubes were shaken thoroughly by inversion and allowed to sit at room temperature for 3 hours, after which the DNA precipitates were spun down in a microcentrifuge at 13,000 rpm for ten minutes. The supernatant from each tube was discarded and the pellets were rinsed with 70% ethanol once. The 70% ethanol was aspirated off and the DNA pellets were allowed to air-dry. Once dry, each pellet was resuspended in 30-50 ul of Tris (10 mM)-EDTA (1 mM) buffer, pH 7.4 and allowed to hydrate and solubilize overnight. 5-7 microliters of each DNA solution was used for each polymerase chain reaction (PCR) procedure.

Two separate PCR procedures were used to analyze transfectants. The first procedure used two primers that were expected to anneal to sites that are both located within the DNA used for transfection. The first primer sequence is homologous to the neomycin resistance cassette of the DNA construct and the second is located approximately 0.5 Kb away, resulting in a short PCR product of 0.5 Kb. This reaction was used to verify that cells surviving G418 selection were resistant as a result of integration of the DNA construct.

Because only a small percentage of transfectants would be expected to contain a DNA integration in the desired location (the Mu locus), another pair of primers was used to determine not only that the DNA introduced was present in the genome of the transfectants but also, that it was integrated in the desired location. The PCR procedure used to detect appropriate integration was performed using one primer located within the neomycin resistance cassette of the DNA construct and one primer that would be expected to anneal over 1.8 Kb away, but only if the DNA had integrated at the appropriate site of the IgM locus (since the homologous region was outside the region included in the DNA construct used for transfection). The primer was designed to anneal to the DNA sequence immediately adjacent to those sequences represented in the DNA construct if it were to integrate in the desired location (DNA sequence of the locus, both within the region present in the DNA construct and adjacent to them in the genome was previously determined).

Using these methods, 135 independent 35 mm plates were screened for targeted integration of the DNA construct into the appropriate locus. Of those, DNA from eight plates were determined to contain an appropriately targeted DNA construct and of those, three were selected for use in nuclear transfer procedures. Those cells lines were designated as “8-1C”, “5-3C”. and “10-1C” Leftover blastocysts not used for transfer into recipient cows were used to extract DNA which was subjected to additional PCR analysis. This analysis was effective using a nested PCR procedure using primers that were also used for initial screening of transfected lines.

As noted above, three cell lines were generated using the gene targeting construct designed to remove exons 1-4 of the mu locus. These lines all tested positive for targeted insertions using a PCR based test and were used for nuclear transfers. Leftover blastocysts resulting from those nuclear transfers were screened by PCR testing the appropriately targeted construct. The following frequencies of positive blastocysts were obtained:

| | |
|---|---|
| Cell Line 8-1C: | 6/8 |
| Cell Line 10-1C: | 2/16 |
| Cell Line 5-3C: | 0/16 |

Although at forty days of gestation, 11 total pregnancies were detected by ultrasound, by day 60, 7 fetuses had died. The remaining 4 fetuses were processed to regenerate new fetal fibroblasts and remaining organs were used to produce small tissue samples for PCR analysis. The results of the analyses are below:
Line 8-1C: two fetuses, one fetus positive for targeted insertion by PCR
Line 10-1C: one fetus, positive for targeted insertion by PCR
Line 5-3C: one fetus, negative for targeted insertion by PCR

Surprisingly, although the frequency of 10-1C blastocysts testing positive for targeted insertion was only 2/16, the one viable 60-day fetus obtained from that cell line was positive as determined by PCR. A positive fetus from 8-1C was also obtained. Southern blot analysis of DNA of all tissue samples is being effected to verify that the construct not only targeted correctly at one end (which is determined by PCR of the shorter region of homology present in the original construct) but also at the other end. Based on results to date, it is believed that two heavy chain knockout fetuses from two independent integration events have been produced. Also, since these fetuses were derived from two different lines, at least one is likely to have integrated construct correctly at both ends. Once the Southern blot analyses have confirmed appropriated targeting of both ends of targeting construct, further nuclear transfers will be performed to generate additional fetuses which will be carried to term.

Nuclear Transfer and Embryo Transfer:

Nuclear transfers were performed with the K/O cell line (8-1-C (18)) and eight embryos were produced. A total of six embryos from this batch were transferred to three disease free recipients at Trans Ova Genetics (“TOG”).

Frozen embryos have been transferred to ten disease free recipients to obtain disease free female fibroblast cell lines. Fetal recoveries will be scheduled after confirming the pregnancies at 35-40 days.

Pregnancy Diagnosis and Fetal Recovery:

Pregnancy status of the eighteen recipients transferred with cloned embryos from knockout fetal cells was checked by ultrasonography.

| Clone ID | No of recips transferred | Pregnancy at 40 days (%) |
|---|---|---|
| 8-1-0C | 5 | 4 (80) |
| 10-1-C | 6 | 4 (67) |
| 5-3-C | 5 | 3 (60) |
| Total | 16 | 11 (69) |

Pregnancy Diagnosis:

Pregnancy status of the three recipients transferred with cloned embryos from knockout cells (8-1C) was checked, one was open and the other two have to be reconfirmed next month.

Pregnancy status said 28 recipients transferred with cloned embryos from cells containing hchr.14fg was checked by ultrasonography.

| | No of recips | Pregnancy at 40 days |
|---|---|---|
| Clone ID | transferred | (%) |
| 2-1 | 08 | 03 (38) |
| 4-2 | 10 | 00 (00) |
| 40-1 | 05 | 00 (00) |
| 4-1 | 03 | 01 (33) |
| 2-1 | 02 | 01 (50) |
| Total | 28 | 05 (18) |

The pregnancy rates are much lower than anticipated. This is believed to be attributable to extremely abnormally hot weather during embryo transfer.

Fetal Recoveries and Establishment of Cell Lines

Eleven pregnancies with the K/O embryos at 40 days were obtained. Four life fetuses were removed out of these at 60 days. Cell lines were established from all four and cryopreserved for future use (Table 2). Also we collected and snap frozen tissue samples from the fetuses and sent them to Hematech molecular biology laboratory for PCR/Southern blot analysis.

All four of the cell lines represented in Table 2 are male. In order to secure female, cell line, cell lines were established not cryopreserved for future establishment of K/O cells from the fetuses (six) collected at 55 days of gestation from the pregnancies established at Trans Ova Genetics with disease free recipients. (Table 3). Recently, the existence confirmed the question of a female cell line containing a mu knockout was confirmed. This female cell line will be used to produce cloned animals which will be mated with animals generated from the male cell lines, and progeny screened for those that contain the double mu knockout.

### Introduction of Hematopoietic Stem Cells Into Transgenic Bovine IgM Knockout

Human hematopoietic stem cells (HSCs) are obtained from peripheral blood, cord blood or bone marrow. The preferred choice is cord blood. Crude cord blood fractions can be separated by centrifugation. To remove hemolyzed blood the cells are pelleted and resuspended in a buffer or the cord blood fracture can be centrifuged over a ficoll gradient separating out the hemolyzed blood, the intact RBCs and white blood fraction. Additionally, HSCs can be obtained after separation based on the CD34 cell surface marker. While the CD34 marker is not unique to HSCs, it is found in a small population of cells that contain HSCs. Approximately 1 million cells (in a volume of about 0.2 to 0.0 ml of buffer) from the crude fractions or considerably fewer (thousands) from a CD34 enriched fraction are injected into the peritoneal cavity of a 75 to 110 day bovine fetus.

The injection procedure comprises making a flank incision into a pregnant cow. The gravid fetus is exposed through the excision. The fetal abdominal area is located by palpitation and by use of an ultrasound probe. An 18 gauge needle attached to a ICC syringe is inserted into the abdominal area and solution of HSCs injected. The fetus is then placed back into the abdominal cavity of the cow and the incision sutured. It is anticipated that these animals upon birth will have a human immune system, at least with respect to T and B cells.

### EXAMPLE 2

Derivation of RAG-2 Knockout Fetuses

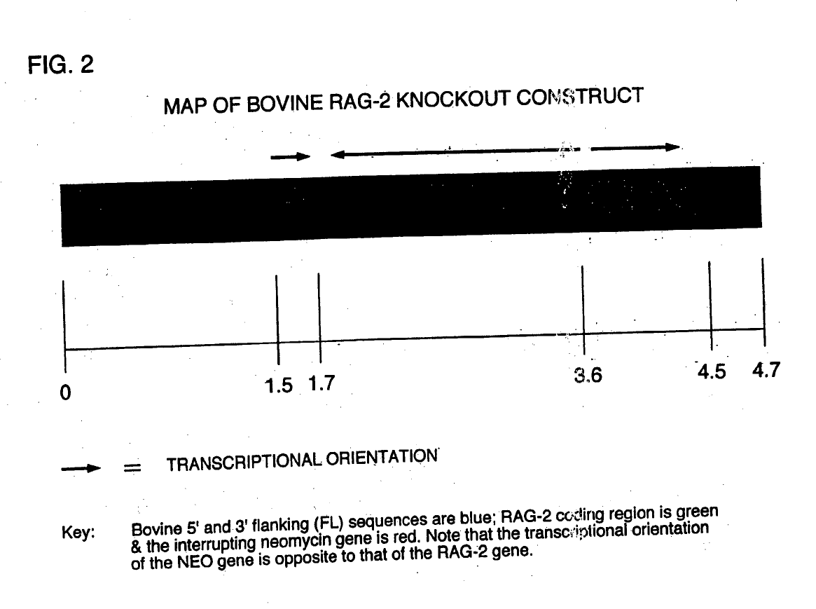

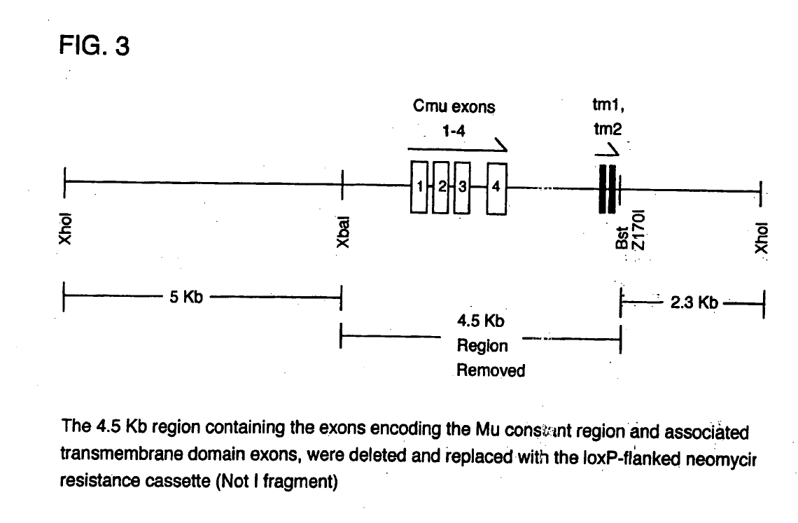

The bovine RAG-2 gene along with 3′ and 5′ flanking sequences was cloned from a bovine lambda ZapII genomic library and used to make the construct, BOVRAG-2-KO, which is shown schematically in FIG. 1. The sequence of bovine rag-2 is contained in FIG. 2. Two versions of this construct have been made. One contains a gene encoding neomycin phosphotransferase (neo) as the selectable marker and the other has puromycin-N-acetyl transferase (puro) as the selectable marker. The construct was introduced into bovine fetal fibroblasts by electroporation using standard techniques (Morrison, S. L., Current Protocols in Immunology, Supplement 12:10.17.10 (1998)). Following electroporation, the cells were washed in complete medium (Alpha MEM supplemented with 10% fetal calf serum penicillin 100 IU/ml, streptomycin 100 IU/ml), resuspended to a concentration of 1X10⁵ cells/ml and distributed in 0.1 ml aliquots to the wells of 96-well culture plates. After 24 hours of incubation, an additional 0.1 ml of 2× selective medium (complete medium+G418 or puromycin, depending on which selectable marker is contained in the constructed) is added. The resistant clones that emerge are screened by PCR to determine which contain construct-mediated disruptions of the RAG-2 gene.

A line of cells derived from a single colony of cells which contains a confirmed RAG-2 gene disrupted by homologous recombination with the BOVRAG-2-KO construct is used to produce donors for nuclear transfer (NT). Nuclear transfer is conducted according to the procedures in Cibelli, J. B. et al, Science 280:1256 (1998). Briefly, oocytes are matured in vitro, stripped of cumulus cells and enucleated at about 18 to 20 hours post maturation (hpm). At about 24 hpm, an individual RAG-2-KO fibroblast are placed in the pervitelline space of a recipient oocyte and fused by electrofusion using a pulse of 120 volts for 15 μsec gap chamber. At around 26 hpm, activation of the NT unit is accomplished by a suitable procedure such as a 4 minute exposure to ionomycin (5 μM) in TL-HEPES supplemented with 1 mg/ml BSA and then washed for 5 minutes in TL-HEPES supplemented with 30 mg/ml BSA. Throughout the ionomycin treatment, NT units are also exposed to 2 mM DMAP. Following the wash, NT units are then transferred into a microdrop of culture medium containing 2 mM DMAP and cultured at 38.5° C. in 5% CO₂ for 4 or 5 hours. Alternatively, activation is effected using cycloheximide and cytochalasin D procedure described infra. Embryos are washed and placed in medium plus 10% FCS and 6 mg/ml BSA in four well plates containing a confluent feeder layer of mouse embryonic fibroblasts. The NT units are then cultured for three more days at 38.5° C. and 5% CO₂. Culture medium is changed every 3 days until 5 to 8 days after activation. Blastocyst and later stage NT embryos are used to produce transgenic animals by transfer into recipient females.

### EXAMPLE 3

Transplantation of Human HSC-enriched Cells into RAG-2-KO Bovine Fetuses.

Populations of human cells enriched for human hematopoietic cells enriched for CD34+ cells will be obtained by standard procedures. They will be introduced into the fetus using an ultrasound guided transvaginal injection method. One arm is inserted into the rectum and is used to manipulate the fetus. The peritoneal cavity of the fetus is located using the ultrasound probe inserted into the vagina. The vaginal probe is moved adjacent to the fetus and an injection needle is extended beyond the probe holder and into the fetus for cell injection. Alternatively, the umbilical cord is held in position by rectal palpation and the needle is inserted into the umbilical artery. The methods are similar to those used for collection of amniotic samples or for ovarian follicle aspirations.

### EXAMPLE 4

Demonstration of Exclusive Production of Polyclonal Human Ig in RAG-KO/human HSC-enriched Transplanted Cattle.

Blood obtained from RAG-KO/enriched-HSC transplanted calves will be subjected to species-specific ELISA to determine if the animals are producing exclusively human Ig or if some bovine Ig is produced. In addition, Ig will be precipitated from each serum sample by mixing with an equal volume of saturated ammonium sulfate. After collection, the precipitate will be dissolved in 5 ml or PBS (pH, 7.2) and dialyzed overnight. The dialyzate will be passed over a column of CNBr-Sepharose to which polyclonal rabbit anti-human Ig has been conjugated. After binding Ig from the serum, the column will be washed with 5 to 10 column volumes of PBS and then sequentially eluted with successive passages of 5 column volumes of following series of buffers: pH 7.0, 0.05 M sodium phosphate; pH5.5, 0.05 sodium Citrate; pH 4.3, 0.5 M sodium acetate; pH 2.3, 0.5 M glycine. Each of the fractions eluted will be checked by bovine and human Ig specific ELISA to verify the presence of human Ig and the absence of bovine Ig.

After its validation as human Ig by ELISA, each purified human Ig sample will be subjected to western blot analysis with class-specific anti-human Ig antibodies and to isoelectric focusing. The western blot analysis will determine the range of different human Ig classes produced and isoelectric focusing will demonstrate that the antibody is polyclonal. With regard to human Ig class, the classes detected by western blotting will vary with the age of the animal. Newborns will likely show a predominance of human Ig, but older calves will be expected to produce various IgG subclasses and IgA in addition to IgM.

### EXAMPLE 5

Demonstration that immunization of RAG-KO/enriched calves respond to immunization of antigen-specific human antibody.

At 60 days of age, RAG-KO/enriched-HSC calves are immunized with tetanus toxoid and the anti-tetanus toxin antibody titer is determined at weekly intervals for 4 weeks following immunization. ELISA using rabbit anti-human antibody as second step detecting reagents will be used to demonstrate that the anti-tetanus antibody response is human antibody. To confirm that the anti-tetanus response is comprised of exclusively human Ig, control experiments using anti-bovine antibody are performed in parallel.

1. A method for producing a cloned ungulate wherein the expression of both copies of a gene essential for B cell production has been knocked out, selected from the group consisting of Igα, IgM, E2A, EBF, BSAP, rag-1 and rag-2, which comprises the following steps:
(i) producing a male and/or female ungulate cell wherein the expression of one or both copies of the Igα, E2A, EBF, BSAP, IgM heavy chain, rag-1 and/or rag-2 gene has been eliminated by targeted disruption;
(ii) using said cell or DNA therefrom as a donor for nuclear transfer by fusing or inserting said donor cell or nucleus into an oocyte or blastomere, which is enucleated before or after transfer, activating the resulting nuclear transfer unit and/or the oocyte prior or simultaneous to nuclear transfer and culturing in a suitable medium to produce a nuclear transfer embryo;
(iii) introducing said nuclear transfer embryo into a female ungulate; and
(iv) obtaining a cloned fetus or animal ungulate that expresses the genotype of the donor differentiated cell, in which one or both copies of the IgM (mu) chain, Igα, E2A, EBF, BSAP, rag-1 and/or rag-2 gene have been eliminated: and
(v) optionally, mating said cloned male or female ungulate with another cloned female ungulate wherein one copy of the IgM, rag-1 or rag-2 gene has been knocked out and selecting progeny wherein both copies of the Igα, E2A, EBF, BSAP, IgM, rag-1 or rag-2 genes have been knocked out.

2. The method of claim 1, wherein the expression of both copies of the E2A,-Igα, EBR, BSAP, IgM heavy chain (mu), rag-1 and/or rag-2 gene is eliminated, by a three-step process comprising the following steps:
(i) a desired ungulate cell is contacted with a DNA construct that provides for targeted deletion or inactivation of said Igα, IgM (mu), E2A, EBF, BSAP, rag-1 or rag-2 gene by homologous recombination;
(ii) the resulting differentiated cell or DNA therefrom, wherein the expression of one copy of the Igα, IgM, EBF, E2R, BSAP, rag-1 and/or rag-2 gene has been knocked out, is used as a nuclear transfer donor and is fused or inserted into an enucleated oocyte;
(iii) the resulting nuclear transfer unit is allowed to develop into an embryo, and a cell is obtained from this embryo and is contacted with a second DNA construct under conditions that results in the elimination of the expression of the other (second) copy of the IgM, Igα, E2A, EBF, BSAP, rag-1 and/or rag-2 gene; by homologous recombination; and
(iv) the resulting cell, in which both copies of the Igα, IgM (mu), E2A, EBF, BSAP rag-1 and/or rag-2 gene have been knocked out, is used as a nuclear donor for nuclear transfer by fusing or inserting said donor cell or DNA therefrom into an enucleated oocyte or blastomere, activating the resultant nuclear transfer unit after oocyte prior to nuclear transfer, and culturing in a suitable medium to produce a nuclear transfer embryo which does not express E2A, EBF, BSAP, Igα, IgM heavy chain, rag-1 or rag-2.

3. The method of claim 1, wherein the ungulate cell used for homologous recombination is a differentiated cell derived from ectoderm, mesoderm or endoderm.

4. The method of claim 1, wherein the donor differentiated cell is a fibroblast cell.

5. The method of claim 1, wherein the cloned ungulate is selected from the group consisting of bovines, pigs, horses, sheep, buffalo and goats.

6. The method of claim 1, wherein the differentiated cell of (i) is produced by sequentially contacting said cell with two knockout constructs which in combination provide for knockout of both copies of the Igα, E2A, EBF, BSAP, IgM, rag-1 and/or rag-2 genes.

7. The method of claim 6, wherein the said two knockout constructs comprise different selectable markers thereby providing for the selection of cells wherein both copies of the IgM heavy chain, rag-1 and/or rag-2 are eliminated.

8. The method of claim 1, wherein said method further comprises the step of introducing the double knockout embryo of (iv) into a female ungulate in order to produce a fetus or live offspring.

9. The method of claim 8, wherein the hematopoietic stem cells are introduced into said cloned fetus while in utero or said live offspring shortly before or after-birth.

10. The method of claim 9, wherein the human hematopoietic stem cells are introduced into said cloned ungulate within about 48 hours to one week before or after birth.

11. The method of claim 9, wherein said hematopoietic stem cells become stably engrafted and result in the formation of functional human B and T lymphocytes.

12. The method of claim 11, wherein human B and T lymphocytes are isolated from the cloned ungulate.

13. The method of claim 11, wherein said human B cells produce human immunoglobulins.

14. The method of claim 11, wherein human immunoglobulins are isolated from the cloned ungulate.

15. A transgenic ungulate wherein both copies of the IgM heavy chain (mu), rag-1 and/or rag-2 gene have been knocked out.

16. A transgenic ungulate according to claim 15 which is selected from the group consisting of bovine, pig, sheep, goat, horse and buffalo.

17. The transgenic ungulate of claim 16 which is a bovine.

18. The transgenic bovine of claim 17 which comprises stably engrafted hematopoietic stem cells of a different species than bovine.

19. The transgenic bovine of claim 18 which comprises human hematopoietic stem cells.

20. The transgenic bovine of claim 18 which comprises canine, feline, murine or primate hematopoietic stem cells.

21. The transgenic ungulate of claim 15 wherein the expression of both copies of the IgM heavy chain gene (mu) have been knocked out.

22. The transgenic ungulate of claim 21 which is a bovine.

23. A method of making non-bovine antibodies from a bovine completely isolating antibodies from the serum of a transgenic bovine according to claim 18.

24. The method of claim 23 wherein said antibodies are human.

25. A method of producing hybridomas comprising:
(1) obtaining B cells from a transgenic bovine according to claim 21;
(2) fusing said cells with an immortal cell to produce a hybridoma cell line.

26. The method of claim 25 wherein said B cells are human B cells.

27. The method of claim 25 wherein said transgenic bovine is immunized with a desired antigen prior to isolation of B cells treatment.

28. The method of claim 27 wherein said B cells are human.

29. A method of expanding human B cells comprising engrafting human hematopoietic stem cells into a transgenic ungulate according to claim 21; and allowing said human hematopoietic stem cell to become stably engrafted and expand in said transgenic ungulate.

30. The method of claim 29, which further comprises recovering said human B and T cells from said transgenic ungulate.

31. A method for maintaining desired tissues, organs or cells in vivo comprising:
(i) engrafting desired allogeneic or xenogeneic tissues in a transgenic ungulate according to claim 15; and
(ii) incubating said tissue in said animal.

32. The method of claim 31 wherein said tissue comprises skin, heart, lung, pancreatic, liver or kidney tissue, cells or organs.
